(19) United States
(12) Patent Application Publication
(10) Pub. No.: US 2015/0034528 A1
(43) Pub. Date: Feb. 5, 2015

(54) PARAFFIN WAX

(71) Applicants: Arlène Marcellina Pouw, Houston, TX (US); Pascal Edzo Armin Rots, Doha (QA); Antonius Adrianus Maria Roovers, Singapore (SG); Lip Piang Kueh, Bintulu (MY); Jelle Sietsma, Amsterdam (NL); Iris Teo Jin Li, Kuala lumpur (MY)

(72) Inventors: Arlène Marcellina Pouw, Houston, TX (US); Pascal Edzo Armin Rots, Doha (QA); Antonius Adrianus Maria Roovers, Singapore (SG); Lip Piang Kueh, Bintulu (MY); Jelle Sietsma, Amsterdam (NL); Iris Teo Jin Li, Kuala lumpur (MY)

(21) Appl. No.: 14/354,944

(22) PCT Filed: Oct. 31, 2012
(86) PCT No.: PCT/EP2012/071564

§ 371 Date: Apr. 29, 2014

(30) Foreign Application Priority Data
Nov. 1, 2011 (EP) 11187378.2

(51) Int. Cl. C09K 5/06; C07C 9/22; C07C 9/14

(52) U.S. Cl. CPC C09K 5/063; C07C 9/14; C07C 9/22
USPC 208/20

(57) ABSTRACT

The present invention provides a Fischer-Tropsch derived paraffin wax comprising paraffins having from 9 to 24 carbon atoms. In another aspect the present invention provides a thermal energy storage material comprising a Fischer-Tropsch derived paraffin wax comprising paraffins having from 9 to 24 carbon atoms, which Fischer-Tropsch derived paraffin wax has a melting point in the range of 15 to 40° C.

The present invention provides a Fischer-Tropsch paraffin wax and a thermal energy storage material comprising the paraffin wax. Furthermore, the present invention provides the use of a paraffin wax as phase change material in thermal energy storage applications.

Paraffin wax may be obtained by various processes. U.S. Pat. No. 2,692,835 discloses a method for deriving paraffin wax from crude oil. Also, paraffin wax may be obtained using the so called Fischer-Tropsch process. An example of such process is disclosed in WO 2002/102941, EP 1 498 469 and WO 2004/009739.

It has now surprisingly been found that specific Fischer-Tropsch derived paraffin waxes can be advantageously used in thermal energy storage materials.

To this end the present invention provides a Fischer-Tropsch derived paraffin wax comprising paraffins having from 9 to 24 carbon atoms, which Fischer-Tropsch derived paraffin wax has a melting point in the range of 15 to 40° C.

An advantage of the present invention is that the paraffin wax has a surprisingly high latent heat, which high latent heat results in the reduction of the amount of paraffin wax needed in a storage material for any particular low-temperature thermal energy storage application.

The Fischer-Tropsch derived paraffin wax according to the present invention is derived from a Fischer-Tropsch process. Fischer-Tropsch derived paraffin wax is known in the art. By the term “Fischer-Tropsch derived” is meant that a paraffin wax is, or is derived from, a synthesis product of a Fischer-Tropsch process. A Fischer-Tropsch derived paraffin wax may also be referred to as a GTL (Gas-to-Liquids) paraffin wax. An example of a Fischer-Tropsch process is given in WO 2002/102941, EP 1 498 469 and WO 2004/009739, the teaching of which is incorporated by reference.

The Fischer-Tropsch derived paraffins are primarily n-paraffins. Preferably, as referenced below, the Fischer-Tropsch derived wax according to the present invention comprises more than 90 wt % of n-paraffins, preferably more than 95 wt % of n-paraffins.

According to the present invention, the Fischer-Tropsch derived paraffin wax comprises paraffins having from 9 to 24 carbon atoms; the Fischer-Tropsch derived paraffin wax comprises preferably at least 70 wt %, more preferably at least 85 wt %, more preferably at least 90 wt %, more preferably at least 95 wt %, and most preferably at least 98 wt % of Fischer-Tropsch derived paraffins having 9 to 24 carbon atoms based on the total amount of Fischer-Tropsch derived paraffins, preferably based on the amount of Fischer-Tropsch derived paraffins having from 9 to 30 carbon atoms.

Suitably, the kinematic viscosity at 40° C. (according to ASTM D445) of the Fischer-Tropsch derived paraffin wax according to the present invention is above 3.0 cSt, preferably above 4.0 cSt, more preferably above 4.5 cSt. Typically, the kinematic viscosity at 40° C. (according to ASTM D445) of the Fischer-Tropsch derived paraffin wax according to the present invention is below 20 cSt, preferably below 15 cSt, more preferably below 10 cSt.

Also, the kinematic viscosity at 100° C. (according to ASTM D445) of the paraffin wax is above 0.5 cSt, preferably above 1.0 cSt, more preferably above 1.5 cSt. Typically, the kinematic viscosity at 100° C. (according to ASTM D445) of the paraffin wax is below 15 cSt, preferably below 10 cSt, more preferably below 5 cSt.

Further, the paraffin wax preferably has a density at 40° C. (according to ASTM D1298) from 0.60 to 0.85 kg/m³, more preferably from 0.70 to 0.80 kg/m³, and most preferably from 0.75 to 0.77 kg/m³.

Preferably, the density at 15° C. (according to ASTM D1298) of the paraffin wax is from 0.65 to 0.90 kg/m³′ more preferably from 0.70 to 0.85, more preferably from 0.75 to 0.80, and most preferably from 0.77 to 0.80 kg/m³.

It is preferred that the specific heat capacity (according to ASTM E 1269-05) of the Fischer-Tropsch derived paraffin wax according to the present invention is in the range of 2.10 to 2.40 J/g° C., more preferably in the range of 2.15 to 2.40 J/g° C., more preferably in the range of 2.15 to 2.35 J/g° C., and most preferably in the range of 2.18 to 2.30 J/g° C. This relatively high specific heat capacity of the Fischer-Tropsch derived wax is of advantage as it will be able to absorb and store an high amount of heat per degree in temperature.

It is especially preferred that the Fischer-Tropsch derived paraffin wax according to the present invention has a latent heat (according to ASTM E793 via Mettler Toledo Differential Scanning Calorimetry (DSC)) between 150 and 220 J/g, preferably between 160 and 210 J/g, more preferably between 180 and 210 J/g. As the latent heat of the Fischer-Tropsch derived paraffin wax according the present invention is surprisingly high, this wax may advantageously be used as phase change materials in thermal energy storage applications, as discussed below.

In a first embodiment the Fischer-Tropsch derived paraffin wax according to the present invention comprises a major amount (i.e. >50 wt %) of Fischer-Tropsch derived paraffins having from 14 to 20, preferably from 16 to 18 carbon atoms; preferably the amount of Fischer-Tropsch paraffins having from 16 to 18 carbon atoms is at least 70 wt %, more preferably at least 75 wt %, more preferably at least 80 wt %, more preferably at least 85 wt %, more preferably at least 90 wt %, and most preferably at least 95 wt % based on the total amount of Fischer-Tropsch paraffins having from 9 to 24 carbon atoms, preferably from 14 to 20 carbon atoms.

Suitably, the Fischer-Tropsch derived paraffin wax comprising Fischer-Tropsch derived paraffins having from 9 to 24 carbon atoms, preferably 14 to 20 carbon atoms, and more preferably 16 to 18 carbon atoms, has a melting point (according to ASTM E794) in the range of 10 to 50° C., preferably in the range of 15 to 40° C., more preferably in the range of 15 to 32° C., more preferably in the range of 15 to 30° C., more preferably in the range of 20 to 30° C., more preferably in the range of 20 to 25° C., more preferably in the range of 20 to 24° C., and most preferably in the range of 21 to 23° C.

Preferably, in the first embodiment of the present invention a Fischer-Tropsch derived paraffin wax comprising paraffins having at least 85 wt % of 16 to 18 carbon atoms, based on the total amount of Fischer-Tropsch derived paraffins having from 9 to 24 carbon atoms, preferably 14 to 20 carbon atoms, has a melting point (according to ASTM E794) in the range of 21 to 23° C. Also, the Fischer-Tropsch derived paraffin wax comprising paraffins having at least 85 wt % of 16 to 18 carbon atoms, based on the total amount of Fischer-Tropsch derived paraffins having from 9 to 24 carbon atoms, preferably 14 to 20 carbon atoms, has a latent heat between 180 and 210 J/g.

In a second embodiment the Fischer-Tropsch derived paraffin wax according to the present invention comprises a major amount (i.e. >50 wt %) of Fischer-Tropsch derived paraffins having from 16 to 22, preferably 18 to 20 carbon atoms; preferably the amount of Fischer-Tropsch derived paraffins having 18 to 20 carbon atoms is at least 65 wt %, more preferably at least 70 wt %, more preferably at least 75 wt %, more preferably at least 80 wt %, more preferably at least 85 wt %, more preferably at least 90 wt %, and most preferably at least 95 wt % based on the total amount of Fischer-Tropsch derived paraffins having from 9 to 24 carbon atoms, preferably 16 to 22 carbon atoms.

Suitably, the Fischer-Tropsch derived paraffin wax comprising Fischer-Tropsch derived paraffins having from 16 to 22, preferably 18 to 20 carbon atoms has a melting point (according to ASTM E794), in the range of 10 to 50° C., preferably in the range of 15 to 40° C., more preferably in the range of 15 to 32° C., more preferably in the range of 15 to 30° C., more preferably in the range of 20 to 30° C., more preferably in the range of 25 to 30° C., and most preferably in the range of 26 to 28° C.

Preferably, in the second embodiment of the present invention a Fischer-Tropsch derived paraffin wax comprising paraffins having at least 80 wt % of 18 to 20 carbon atoms, based on the total amount of Fischer-Tropsch derived paraffins having from 9 to 24 carbon atoms, preferably 16 to 22 carbon atoms, has a melting point in the range of 26 to 28° C. Also, the Fischer-Tropsch derived paraffin wax comprising paraffins having at least 80 wt % of 18 to 20 carbon atoms based on the total amount of Fischer-Tropsch derived paraffins having from 9 to 24 carbon atoms, preferably 16 to 22 carbon atoms, has a latent heat of between 180 and 210 J/g.

Known to those skilled in the art is that the temperature and pressure at which the Fischer-Tropsch process is conducted influences the degree of conversion of synthesis gas into hydrocarbons and impacts the level of branching of the paraffins (thus amount of isoparaffins). Typically, the process for preparing a Fischer-Tropsch derived wax may be carried out at a pressure above 25 bara. Preferably, the Fischer-Tropsch process is carried out at a pressure above 35 bara, more preferably above 45 bara, and most preferably above 55 bara. A practical upper limit for the Fischer-Tropsch process is 200 bara, preferably the process is carried out at a pressure below 120 bara, more preferably below 100 bara.

The Fischer-Tropsch process is suitably a low temperature process carried out at a temperature between 170 and 290° C., preferably at a temperature between 180 and 270° C., more preferably between 200 and 250° C.

The amount of isoparaffins is suitably less than 20 wt % based on the total amount of paraffins having from 9 to 24 carbon atoms, preferably less than 10 wt %, more preferably less than 7 wt %, and most preferably less than 4 wt %.

Suitably, the Fischer-Tropsch derived paraffin wax according to the present invention comprises more than 90 wt % of n-paraffins, preferably more than 95 wt % of n-paraffins. Further, the paraffin wax may comprise iso-paraffins, cyclo-alkanes and alkyl benzene.

The Fischer-Tropsch process for preparing the Fischer-Tropsch derived wax according the present invention may be a slurry Fischer-Tropsch process, an ebullated bed process or a fixed bed Fischer-Tropsch process, especially a multitubular fixed bed. The product stream of the Fischer-Tropsch process is usually separated into a water stream, a gaseous stream comprising unconverted synthesis gas, carbon dioxide, inert gasses and C1 to C3, and a C4+ stream.

The full Fischer-Tropsch hydrocarbonaceous product suitably comprises a C1 to C200 fraction, preferably a C3 to C200 fraction, more preferably a C4 to C150 fraction.

Suitably, the Fischer-Tropsch derived paraffin wax according to the present invention is obtained from the Fischer-Tropsch hydrocarbonaceous product by distillation. Commercially available equipment can be used. The distillation may be carried out at atmospheric pressure, but also reduced pressure may be used.

Preferably, the hydrocarbonaceous product stream of the Fischer-Tropsch process, comprising a C3 to C200 fraction, preferably a C4 to C150 fraction is hydrogenated before distillation, in order to remove oxygenates and olefins. Further, such hydrogenated product stream is more stable and less corrosive, making transport and/or storage more easy.

The hydrogenation step is suitably carried out at a temperature between 150 and 325° C., preferably between 200 and 275° C., a pressure between 5 and 120 bar, preferably between 20 and 70 bar.

Lighter fractions of the Fischer-Tropsch product, which suitably comprises C3 to C8 fraction, preferably C4 to C7 fraction, are separated from the Fischer-Tropsch product by distillation thereby obtaining a first Fischer-Tropsch product, which suitably comprises C9 to C200 fraction.

Subsequently, a second Fischer-Tropsch product, which suitably comprises C9 to C24 fraction, is obtained by separation of a heavy fraction, which heavy fraction suitably comprises C25 to C200 fraction, preferably C25 to C150 fraction, from the first Fischer-Tropsch product by distillation. Suitably, the distillation is carried out at a pressure of in between 50 to 70 mbara and at a temperature of from 125 to 145° C. in the top section of the column.

After, a light fraction, which suitably comprises C9 to C13 fraction is separated from the second Fischer-Tropsch product by distillation, thereby obtaining a third Fischer-Tropsch product, which suitably comprises C14 to C24 fraction. It is preferred that the distillation is carried out at a pressure of 500 to 700 mbara and a temperature of 230 to 250° C. in the bottom stripping section of the column.

Hereafter, the Fischer-Tropsch derived paraffin wax according to the present invention is separated from the third Fischer-Tropsch product. Suitably, the distillation is suitably carried out in the rectifying section in the column at a pressure of in between 200 to 250 mbara and at a pressure of 450 to 500 mbara in the stripping section of the column. Also, the distillation is preferably carried out at a temperature of from 200 to 250° C. in the rectifying section of the column.

Preferably, to obtain a Fischer-Tropsch derived paraffin wax comprising paraffins having from 14 to 20 carbon atoms, preferably 16 to 18 carbon atoms from the third Fischer-Tropsch product the temperature in the distillation column is between 220 to 230° C.

Preferably, to obtain a Fischer-Tropsch derived paraffinic wax comprising paraffins having from 16 to 22 carbon atoms, preferably 18 to 20 carbon atoms from the third Fischer-Tropsch product the temperature in the column is between 225 to 240° C.

In another embodiment the process according to the present invention comprises hydrogenation of smaller fractions obtained by distillation of the full hydrocarbonaceaous product. Hydrogenation after distillation avoids the need to hydrogenate a large amount of Fischer-Tropsch product. For example, the second Fischer-Tropsch product, which suitably comprises a C9 to C24 fraction, is hydrogenated in one or more separate fractions before being distilled into the Fischer-Tropsch derived paraffin wax comprising paraffins having from 14 to 24 carbon atoms, preferably 14 to 20 carbon atoms, more preferably 16 to 18 carbon atoms or into a Fischer-Tropsch derived paraffin wax comprising paraffins having from 16 to 22 carbon atoms, preferably 18 to 20 carbon atoms.

In a further aspect, the present invention provides a thermal energy storage material comprising Fischer-Tropsch derived paraffin wax according to the present invention. Typically, the thermal energy storage material may be used in many areas, for instances as the thermal insulation of lines or pipes carrying fluids, in building materials and fabric for attires. Also, the thermal energy storage material may be based on phase change materials (PCM).

PCMs are compounds with a high latent heat, which melt and solidify at certain temperature ranges, and thus are capable of storing or releasing large amounts of energy (heat). The transition from solid to liquid phase (melting process) is an endothermic process, which results in absorption of energy. The material begins to melt upon reaching the phase change temperature. During this melting process the temperature stays almost constant until the melting is finished. The heat stored during melting is the latent heat. Equally, when the phase change process is reversed (from liquid to solid phase), the stored latent heat is released again at a nearly constant temperature. Furthermore, to minimize the physical size of the heat storage device, the latent heat should be as high as possible and the density difference between the solid and liquid should be as small as possible.

An advantage of the use of the paraffin wax as PCM in thermal energy storage material is that the thermal energy storage material has surprisingly high latent heat. This can give a substantial reduction in the quantity of materials required for thermal energy storage.

Suitably, the amount of paraffin wax according to the present invention as PCM in thermal energy storage material is preferably at most 100 wt % and preferably at least 90 wt %, more preferably at least 95 wt %, and most preferably at least 98 wt % based on the total amount of thermal storage material. Further, the thermal storage material may—in addition to the Fischer-Tropsch derived paraffin wax as PCM—conveniently comprise additives such as nucleating agents, anti-oxidant or anti-bacterial agents, corrosion inhibitors or an insoluble filler designed to improve its stability or a solvent designed to control its viscosity, etc.

Accordingly, the present invention provides the use of the paraffin wax according to the present invention as phase change material in thermal energy storage applications.

The person skilled in the art will readily understand that the thermal energy storage material has the similar kinematic viscosity, density, specific heat capacity, latent heat and melting point as the paraffin wax according to the present invention, provided that the amount of paraffin wax used as PCM in the thermal energy storage material is at most 100 wt % and at least 90 wt %, preferably at least 95 wt %, and most preferably at least 98 wt % based on the total amount of thermal energy storage material.

The present invention is described below with reference to the following Examples, which are not intended to limit the scope of the present invention in any way.

### EXAMPLES

### Preparation of Fischer-Tropsch Derived Paraffin Waxes

Two Fischer-Tropsch derived paraffin waxes (Paraffin wax 1 and Paraffin wax 2) were obtained using a Fischer-Tropsch process. To this end, the Fischer-Tropsch product as obtained in Example VII using the catalyst of Example III of WO-A-9934917 was hydrogenated at a temperature between 200 and 275° C. and at a pressure between 20 and 70 bar. The obtained Fischer-Tropsch product comprised a C4 to C150 fraction. After hydrogenation, lighter fractions of the Fischer-Tropsch product, which comprised C4 to C7 fraction, were separated from the Fischer-Tropsch product by distillation, thereby obtaining a first Fischer-Tropsch product.

Subsequently, a second Fischer-Tropsch product was obtained by separation of a heavy fraction, which heavy fraction comprised C25 to C150 fraction, from the first Fischer-Tropsch product by distillation at a pressure of in between 50 to 70 mbara and at a temperature of 140° C. in the top section of the column.

After, a light fraction, which comprised C9 to C13 fraction, was separated from the second Fischer-Tropsch product by distillation at a pressure of 500 to 700 mbara and at a temperature of 230° C., thereby obtaining a third Fischer-Tropsch product, which comprises a C14 to C24 fraction.

Paraffin wax 1 was separated from the third Fischer-Tropsch product at a temperature of 221.4° C. and at a pressure of in between 200 to 250 mbara in the receiving section of the distillation column and at a pressure of in between 450 to 500 mbara in the stripping section of the column. The properties of the obtained Paraffin wax 1 are listed in Table 1.

Paraffin wax 2 was separated from the third Fischer-Tropsch product at a temperature of 227.9° C. and at a pressure of in between 200 to 250 mbara in the receiving section of the distillation column and at a pressure of in between 450 to 500 mbara in the stripping section of the column. The properties of the obtained Paraffin wax 2 are listed in Table 1.

TABLE 1

| | Paraffin wax 1 | Paraffin wax 2 |
|---|---|---|
| carbon range | C14-C20 | C16-C22 |
| amount of C16-C18 [wt %] | 92^{a} | 45^{a} |
| amount of C18-C20 [wt %] | 45^{a} | 84^{a} |
| melting point according | 22.3 | 26.7 |
| to ASTM E794 [° C.] | | |
| density at 15° C. | 0.782 | 0.786 |
| according to ASTM D1298 | | |
| [kg/m³] | | |
| density at 40° C. | 0.769 | 0.770 |
| according to ASTM D1298 | | |
| [kg/m³] | | |
| kinematic viscosity at | 4.0 | 3.6 |
| 40° C. according to ASTM | | |
| D445 [CSt] | | |
| specific heat capacity | n.d. | 2.27 |
| according to ASTM E | | |
| 1269-05 [J/g ° C.] | | |

^{a}The sum of the amounts of [C16-C18] and [C18-C20] in Paraffin waxes 1 and 2 is more than 100 wt % due to the fact that both carbon ranges comprise the amount of paraffin C18.

### Determination of the Latent Heat

### Sample Preparation for DSC Latent Heat Measurements

Paraffin waxes 1 and 2 were prepared for the DSC latent heat measurements comprising the following steps:

a) a sample of the paraffin wax was kept in an oven or in a hot water bath until the sample was fully melted;

b) an empty pan was placed on a balance, tarred to zero;

c) with the aid of a Pasteur pipette the homogenized melted sample was withdrawn into the sample pan and a weight of 0.01 mg was recorded;

d) a lid was put on the sample pan in order to close the sample pan;

e) before analysis the total weight of the sample pan, lid and sample was recorded.

ASTM E793 was followed for determining the latent heat by DSC. The latent heat of Paraffin wax 1 (Example 1) and Paraffin wax 2 (Example 2) were measured with a Mettler Toledo DSC equipped with Julabo intracooler FT100 chiller at heating and cooling rates of 10° C./min.

The melting points of the Paraffin waxes were determined according to ASTM E794.

The latent heat and the melting points of the Paraffin wax 1 (Example 1) and 2 (Example 2) are shown in Table 2.

In order to show the increased latent heat of the Fischer-Tropsch derived paraffin waxes according to the present invention, the following commercially available phase change materials were included as Comparative Examples:
Rubitherm® 20 (RT20; obtainable from DuPont, Meyrin, Switzerland; Comparative Example A)
Rubitherm® 27 (RT27; obtainable from DuPont, Meyrin, Switzerland; Comparative Example B)
Astorphase® 20B (AP20B; obtainable from International Waxes Inc., Pennsylvania, United States; Comparative Example C)
Astorphase® 25 (AP25; obtainable from International Waxes Inc., Pennsylvania, United States; Comparative Example D)

TABLE 2

| | Comp | Comp | | Comp | Comp | |
|---|---|---|---|---|---|---|
| | Exam A | Exam C | Example 1 | Exam B | Exam D | Example 2 |
| carbon | C13-C22 | n.d. | C14-C20 | C17-C20 | n.d. | C16-C22 |
| range | | | | | | |
| melting | 23.2 | 19.7 | 22.3 | 28.5 | 23.4 | 26.7 |
| point | | | | | | |
| [° C.] | | | | | | |
| latent | 113.41 | 110.48 | 197.10 | 131.33 | 127.46 | 207.0 |
| heat | | | | | | |
| [J/g] | | | | | | |
| n- | 98.8 | n.d. | 93.9 | 99.8 | n.d. | 94.2 |
| paraffins | | | | | | |
| [wt %] | | | | | | |

### Discussion

The results in Table 2 show that with Fischer-Tropsch derived Paraffin wax 1 (Example 1) a higher latent heat was obtained compared to RT20 (Comparative Example A) and AP20B (Comparative Example C), which both have similar melting points as Paraffin wax 1.

Similar results were obtained with Fischer-Tropsch derived Paraffin wax 2 (Example 2), with which a higher latent heat was obtained compared to the latent heats of RT27 (Comparative Example B) and AP25 (Comparative Example D).

These observations indicate that a lower amount of the Paraffin wax 1 and Paraffin wax 2 in thermal energy storage materials is required to obtain the same latent heat when compared to the amount of RT20, RT27, AP20B and AP25.

1. A Fischer-Tropsch derived paraffin wax comprising paraffins having from 9 to 24 carbon atoms, which Fischer-Tropsch derived paraffin wax has a melting point in the range of 15 to 40° C.

2. A paraffin wax according to claim 1, wherein the Fischer-Tropsch derived paraffin wax has a kinematic viscosity at 40° C. above 3.0 cSt.

3. A paraffin wax according to claim 1, wherein the Fischer-Tropsch derived paraffin wax has a kinematic viscosity at 100° C. above 0.5 cSt.

4. A paraffin wax according to claim 1, wherein the Fischer-Tropsch derived paraffin wax has a density at 40° C. from 0.60 to 0.85 kg/m³.

5. A paraffin wax according to claim 1, wherein the Fischer-Tropsch derived paraffin wax has a density at 15° C. from 0.65 to 0.90 kg/m³.

6. A paraffin wax according to claim 1, wherein the Fischer-Tropsch derived paraffin wax has a specific heat capacity in the range of 2.15 to 2.35 J/g° C.

7. A paraffin wax according to claim 1, wherein the Fischer-Tropsch derived paraffin wax has a latent heat between 150 and 220 J/g.

8. A paraffin wax according to claim 1, wherein the amount of Fischer-Tropsch derived paraffins having from 16 to 18 carbon atoms is at least 85 wt %.

9. A paraffin wax according to claim 8, wherein the Fischer-Tropsch derived wax has a melting point in the range of 20 to 24° C.

10. A paraffin wax according to claim 1, wherein the amount of Fischer-Tropsch derived paraffins having from 18 to 20 carbon atoms is at least 80 wt %.

11. A paraffin wax according to claim 10, wherein the Fischer-Tropsch derived paraffin wax has a melting point in the range of 25 to 30° C.

12. A thermal energy storage material comprising a paraffin wax according to claim 1.

13. A material according to claim 12, wherein the material comprises at least 90 wt %.

14. A use of paraffin wax according to claim 1 as phase change material in thermal energy storage applications.
